(19) United States
(12) Patent Application Publication
(10) Pub. No.: US 2012/0309821 A1
(43) Pub. Date: Dec. 6, 2012

(54) Use of epigallocatechin-3-gallate for immune regulation

(75) Inventors: Ann Chen, Taipei City (TW); Shuk-Man Ka, Taipei City (TW); Jia-Ming Chang, Taipei City (TW); Jiann-Jyh Huang, Taipei City (TW)

(73) Assignee: NATIONAL DEFENSE MEDICAL CENTER, Taipei City (TW)

(21) Appl. No.: 13/153,088

(22) Filed: Jun. 3, 2011

(51) Int. Cl. A61K 31/353; A61P 13/12; A61P 19/04

(52) U.S. Cl. 514/456

(57) ABSTRACT

The preset invention relates to a method for treating lupus erythematosus, particularly lupus nephritis, comprising administering a subject in need thereof a therapeutically effective amount of epigallocatechin-3-gallate (EGCG) or a pharmaceutically acceptable salt or a physiologically functional derivative, together with one or more pharmaceutically acceptable carriers, diluents or excipients.

### FIELD OF THE INVENTION

The present invention relates to new use of Epigallocatechin-3-gallate (EGCG) for immune regulation, particularly for preventing and treating lupus etythematosus, particularly systemic lupus etythematosus (SLE) or lupus nephritis.

### BACKGROUND OF THE INVENTION

In recent years, oxidative stress has attracted considerable attention because of its potential role in the pathogenesis of systemic lupus erythematosus (SLE). Patients with lupus nephritis, a major cause of mortality and morbidity of SLE, show impaired oxidative status which is associated with the progression of lupus nephritis. Oxidative stress, defined as an excess of pro-oxidant species not counterbalanced by an adequate endogenous and exogenous antioxidant defense system, can induce inflammation by activating nuclear factor-kappaB (NF-κB) and stimulating the subsequent production of pro-inflammatory cytokines and chemokines to promote chronic kidney disease. The nuclear factor E2-related factor 2 (Nrf2) antioxidant signaling pathway plays a major role in cellular defense against oxidative stress, and ablation of the Nrf2 gene has been shown to cause a lupus-like autoimmune nephritis. Several mouse models and microarray assays have highlighted a role of Nrf2 in immune regulation and inflammation. Moreover, disruption of Nrf2 has been reported to cause increased production of proinflammatory cytokines, such as interleukin (IL)-1β, tumor necrosis factor (TNF)-α, and IL-6 in mouse models of traumatic brain injury or acute lung injury, while blockade of oxidative stress has been reported to improve renal inflammation by decreasing interleukin IL-1β and TNF-α expression.

Recent studies have demonstrated that IL-18 levels are associated with the severity of renal damage in patients with SLE and in lupus-prone New Zealand black/white (NZB/W) F1 mice (Shui, H. A. et al. LPS-evoked IL-18 expression in mesangial cells plays a role in accelerating lupus nephritis. Rheumatology (Oxford) 46:1277-1284; 2007) or MLR/lpr mice (Faust, J. et al. Correlation of renal tubular epithelial cell-derived interleukin-18 up-regulation with disease activity in MRL-Faslpr mice with autoimmune lupus nephritis. Arthritis Rheum 46:3083-3095; 2002). NOD-like receptor (NLR) family members are a group of pattern recognition receptors involved in a wide variety of microbial components and danger signals. In response to pathogen-associated molecular patterns or danger signals, a subset of NLRs forms a complex with apoptosis-associated speck-like protein that contains a caspase recruitment domain to activate caspase-1 (Franchi, L. et al. The inflammasome: a caspase-1-activation platform that regulates immune responses and disease pathogenesis. Nat Immunol 10:241-247, 2009), this complex being termed the inflammasome (Martinon, F. et al., The inflammasome: a molecular platform triggering activation of inflammatory caspases and processing of proIL-beta. Mol Cell 10:417-426; 2002.) The NLRP3 inflammasome, one of that being best characterized, controls the activation of caspase-1, which then cleaves pro-IL-1β and pro-IL-18 to form mature IL-1β and IL-18 (Agostini, L. et al. NALP3 forms an IL-1beta-processing inflammasome with increased activity in Muckle-Wells autoinflammatory disorder. Immunity 20:319-325; 2004.) Recently, Vilaysane et al. demonstrated that renal inflammation in a unilateral ureteral obstruction mice model is linked to the NLRP3 inflammasome and overproduction of IL-1β and IL-18 and that the NLRP3 inflammasome plays a role in a variety of human nondiabetic kidney diseases and chronic kidney diseases. (Vilaysane, A. et al. The NLRP3 inflammasome promotes renal inflammation and contributes to CKD. J Am Soc Nephrol 21:1732-1744; 2010.)

Epigallocatechin-3-gallate (EGCG) is the major bioactive polyphenols in green tea. (Singh, R. et al. Green tea polyphenol epigallocatechin-3-gallate: inflammation and arthritis. Life Sci 86:907-918; 2010.) In addition to its well known antioxidant activity and free radical-scavenging capacity, several studies have demonstrated that it has potent anti-inflammatory effects due to its inhibiting NF-κB-mediated inflammatory responses (Melgarejo, E. et al. Targeting of histamine producing cells by EGCG: a green dart against inflammation? J Physiol Biochem 66:265-270; 2010; Ahmad, N. et al. Green tea polyphenol epigallocatechin-3-gallate differentially modulates nuclear factor kappaB in cancer cells versus normal cells. Arch Biochem Biophys 376:338-346; 2000), suppressing T cell activation (Watson, J. L. et al. Immune cell activation and subsequent epithelial dysfunction by Staphylococcus enterotoxin B is attenuated by the green tea polyphenol (−)-epigallocatechin gallate. Cell Immunol 237:7-16; 2005), and increasing the number of Foxp3-expressing regulatory T (Treg) cells (Yun, J. M. et al. Effects of epigallocatechin gallate on regulatory T cell number and function in obese v. lean volunteers. Br J Nutr 103:1771-1777; 2010). Although EGCG has been reported to have renal protective effects in mice bearing the solid Ehrlich ascites carcinoma (El-Mowafy, A. M. et al. Novel chemotherapeutic and renal protective effects for the green tea (EGCG): role of oxidative stress and inflammatory-cytokine signaling. Phytomedicine 17:1067-1075; 2010), in streptozotocin-induced diabetic nephropathy rats (Yamabe, N. et al. Therapeutic potential of (−)-epigallocatechin 3-O-gallate on renal damage in diabetic nephropathy model rats. J Pharmacol Exp Ther 319:228-236; 2006), and in cisplatin-induced nephrotoxicity rats (Sahin, K. et al. Epigallocatechin-3-gallate activates Nrf2/HO-1 signaling pathway in cisplatin-induced nephrotoxicity in rats. Life Sci 87:240-245; 2010). However, none of the prior art references dislcosed the use of EGCG in regulation of immune.

### BRIEF SUMMARY OF THE INVENTION

The present invention provides a new approach for treatment of immune diseases, particularly lupus etythematosus (SLE).

In one aspect, the invention provides a method for regulating immune comprising administering a subject in need thereof a therapeutically effective amount of EGCG or a pharmaceutically acceptable salt or a physiologically functional derivative, together with one or more pharmaceutically acceptable carriers, diluents or excipients.

In another aspect, the invention provides a method for treating or preventing lupus etythematosus comprising administering a subject in need thereof a therapeutically effective amount of EGCG or a pharmaceutically acceptable salt or a physiologically functional derivative, together with one or more pharmaceutically acceptable carriers, diluents or excipients.

In particular, the invention provides a method for preventing or treating systemic lupus erythematosus. On the other hand, the invention provides a method for preventing or treating local Lupus erythematosus. For example, lupus erythematosus is lupus nephritis.

In further aspect, the invention provides a method for preventing the development of lupus nephritis comprising administering a subject in need thereof a therapeutically effective amount of EGCG or a pharmaceutically acceptable salt or a physiologically functional derivative, together with one or more pharmaceutically acceptable carriers, diluents or excipients.

### BRIEF DESCRIPTION OF THE DRAWINGS

For the purpose of illustrating the invention, there are shown in the drawings embodiments. It should be understood, however, that the invention is not limited to the preferred embodiments shown. In the drawings:

FIG. 1 shows the clinical and pathological features in urine protein time-course studies (A), serum blood urea nitrogen (BUN) levels (B) and serum creatinine levels (C) at week 34, and kidney histopathological evaluation by H&E staining at week 34 (D). The arrowheads and arrows indicate crescent formation and periglomerular infiltration, respectively. The scoring of the percentage of glomeruli affected by the indicated parameter is shown in the left lower panel. (E) Kidney IgG deposits in glomeruli at week 34 detected by immunofluorescence staining. Original magnification, 400×. The scoring of the intensity of staining for IgG is shown in the right lower panel. (F) Serum anti-dsDNA antibody levels at week 34. In A, the data are the mean±SEM for six mice per group; the horizontal dashed line is the mean for normal control mice. In the histograms in B-F, the data are the mean±SEM for six mice per group for the disease control mice (black bars), EGCG-treated mice (hatched bars), or normal control mice (white bars). *p<0.05, **p<0.01, ***p<0.005. # Not detectable.

FIG. 2 shows the ROS production in superoxide anion levels at week 34 in the serum (A), urine (B), and kidney (C), kidney in-situ ROS production at week 34 (D) demonstrated by dihydroethidium labeling. The arrows indicate the glomeruli. Original magnification, 400×. The scoring of the percentage of positive nuclei is shown in the right panel. In the histograms, the data are the mean±SEM for six mice per group for the disease control mice (black bars), EGCG-treated mice (hatched bars), or normal control mice (white bars). ***p<0.005.

FIG. 3 shows the Nrf2 signaling pathway and GPx activity in terms of the representative Western blots of nuclear Nrf2 (A) and cytosolic p47^{phox} (B) levels in kidney tissues at week 34, Histone H3 and 13-actin were used as internal controls for nuclear and cytosolic proteins, respectively, the quantification of the Nrf2/histone H3 ratio (C) and p47^{phox}/β-actin ratio (D), and GPx activity in the kidney at week 34 (E). In the histograms, the data are the mean±SEM for six mice per group for the disease control mice (black bars), EGCG-treated mice (hatched bars), or normal control mice (white bars). *p<0.05, **p<0.01, ***p<0.005.

FIG. 4 shows the T cell/macrophage infiltration and NF-κB activation in the kidney by the detection of CD3⁺ T cells (A), F4/80 monocytes/macrophages (B), or NF-κB p65 (C) at week 34 by immunohistochemical staining (Original magnification, 400×, the scoring is shown in the lower panels. gcs, glomerular cross section), and the kidney NF-κB activity at week 34 measured by ELISA (E). In the histograms, the data are the mean±SEM for six mice per group for the disease control mice (black bars), EGCG-treated mice (hatched bars), or normal control mice (white bars). *p<0.05, **p<0.01, ***p<0.005.

FIG. 5 shows the NLRP3 inflammasome activation in terms of the serum levels of IL-1β (A) or IL-18 (B) at week 34 measured by ELISA, the measurement of NLRP3 mRNA at week 34 by real time PCR (C), the representative Western blots of NLRP3 (D), IL-1β (E), IL-18 (F), or caspase-1 (Casp1), in kidney tissues at week 34 (G), the appearance of mature 17 kDa IL-1β, 18 kDa IL-18, and the Casp1 p20 subunit indicate activation. β-actin was used as internal control. (H-K): quantification of the NLRP3/β-actin ratio (H), mature IL-1β/β-actin ratio (I), mature IL-18/β-actin ratio (J), or Casp1/β-actin ratio (K). In the histograms, the data are the mean±SEM for six mice per group for the disease control mice (black bars), EGCG-treated mice (hatched bars), or normal control mice (white bars). *p<0.05, **p<0.01, ***p<0.005.

FIG. 6 shows the systemic cellular immunity and Treg cell function by the percentage of CD3⁺CD69⁺ cells in CD3⁺ splenocytes (A) or CD19⁺CD69⁺ cells in CD19⁺ splenocytes (B) at week 34, the proliferation of isolated Treg cells or responder T (Tresp) cells in response to activation by anti-CD3 antibody (C), and the inhibitory activity of CD4⁺CD25⁺ Treg cells by performing titration studies (D). The data are the mean±SEM for six mice per group. A-C: disease control mice (black bars), EGCG-treated mice (hatched bars), or normal control mice (white bars). *p<0.05, **p<0.01, ***p<0.005.

### DETAILED DESCRIPTION OF THE INVENTION

Unless defined otherwise, all technical and scientific terms used herein have the meaning commonly understood by a person skilled in the art to which this invention belongs. As used herein, the following terms have the meanings ascribed to them unless specified otherwise.

The articles “a” and “an” are used herein to refer to one or more than one (i.e., at least one) of the grammatical object of the article. By way of example, “an element” means one element or more than one element.

The table below shows the abbreviations for some terminologies.

| Abbreviation | Terminology |
|---|---|
| BUN | blood urea nitrogen |
| Cr | Creatinine |
| DHE | Dihydroethidium |
| EGCG | epigallocatechin-3-gallate |
| ELISA | enzyme-linked immunosorbent assay |
| GPx | glutathione peroxidase |
| IHC | Immunohistochemistry |
| IL | Interleukin |
| NF-κB | nuclear factor-kappa B |
| NLR | NOD-like receptor |
| Nrf2 | nuclear factor E2-related factor 2 |
| NZB/W | New Zealand black/white |
| RLU | reactive luminescence units |
| ROS | reactive oxygen species |
| SLE | systemic Lupus erythematosus |
| Tconv | conventional T |
| TNF | tumor necrosis factor |
| Treg | regulatory T |

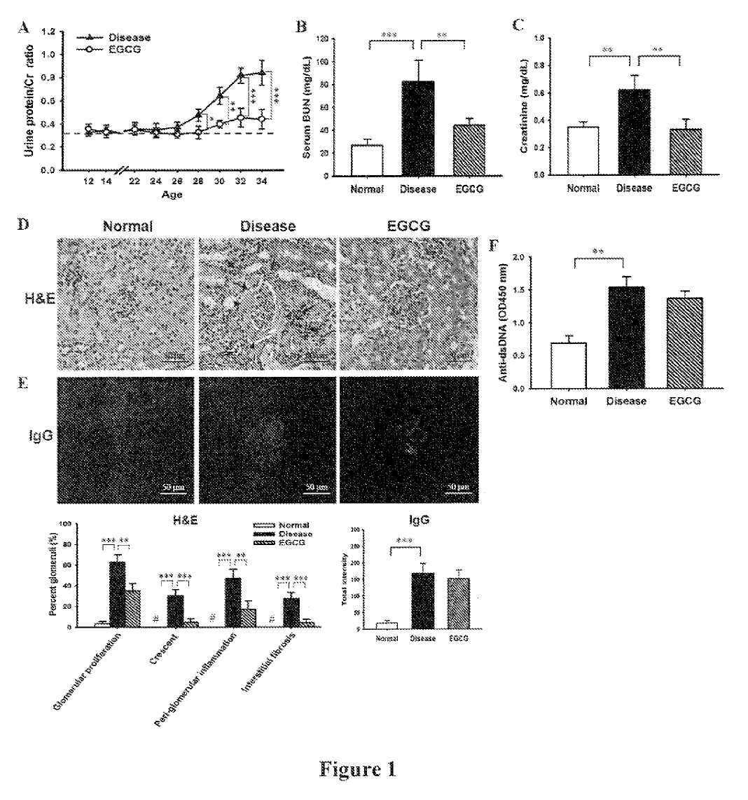

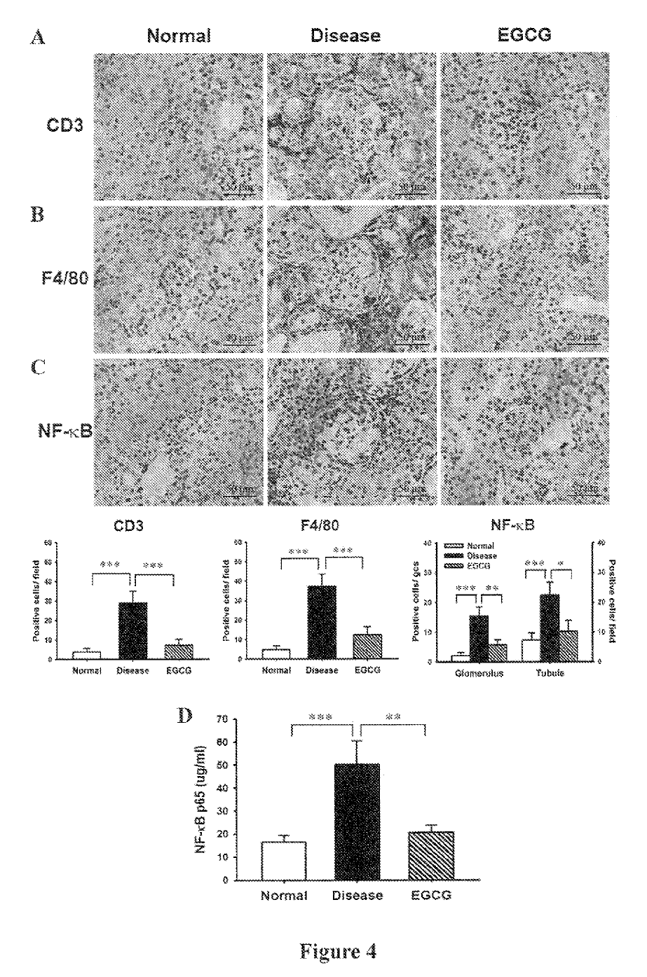

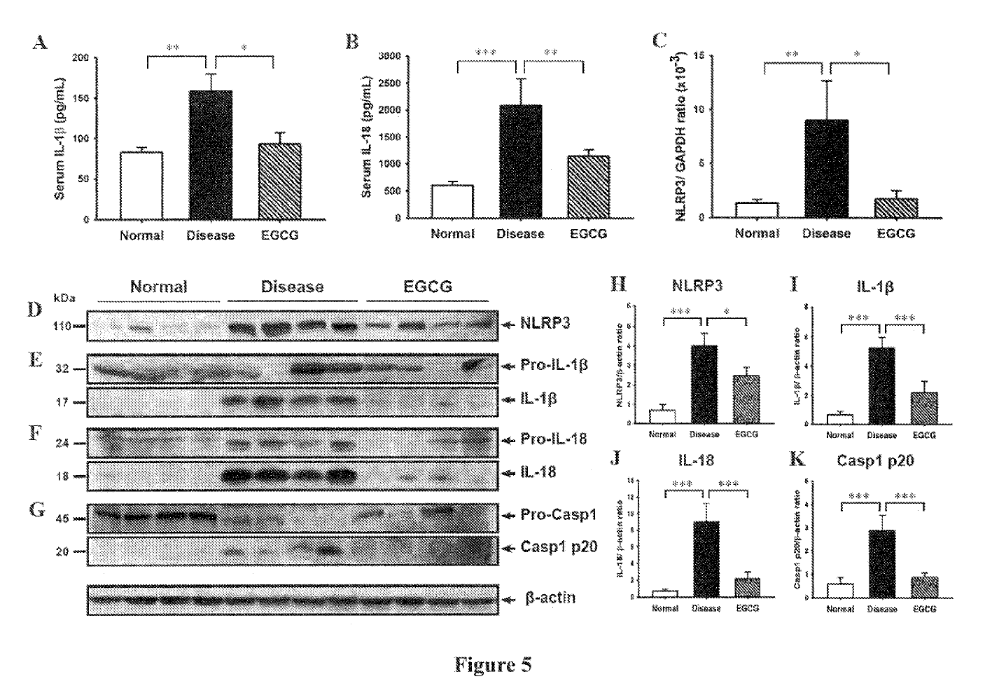

In the present invention, it was unexpectedly found that Epigallocatechin-3-gallate (EGCG) could prevent development of lupus nephritis by administering it to NZB/W F1 lupus-prone mice and demonstrated that it activated the Nrf2 antioxidant pathway, inhibited renal NAPL3, such as those as sown in FIGS. 1E and 1F. Accordingly, EGCG can be used as a selective immune regulator in vivo. In one example of the invention, EGCG was proved to have an effect on inflammasome activity and Treg function thus EGCG provides a protection for kidney from severe pathological lesions. In one example of the invention, EGCG was proved to have an effect on infiltration by T cells and monocytes/macrophages, see FIGS. 4A-4D. In another example of the invention, EGCG was confirmed to have an effect on enhancement of serum levels of IL 1 and IL-18. Furthermore, EGCG could regulate systemic cellular immunity and Treg cell activation, see FIG. 5.

The invention provides a method for regulating immune comprising administering a subject in need thereof a therapeutically effective amount of EGCG or a pharmaceutically acceptable salt or a physiologically functional derivative, together with one or more pharmaceutically acceptable carriers, diluents or excipients.

In particular, the invention provides a method for preventing and treating lupus erythematosus comprising administering a subject in need thereof a therapeutically effective amount of EGCG or a pharmaceutically acceptable salt or a physiologically functional derivative, together with one or more pharmaceutically acceptable carriers, diluents or excipients. For example, the invention provides a method for preventing or treating systemic Lupus erythematosus (SLE).

Furthermore, the present invention provides a method for treating or preventing lupus nephritis comprising administering a subject in need thereof a therapeutically effective amount of EGCG or a pharmaceutically acceptable salt or a physiologically functional derivative, together with one or more pharmaceutically acceptable carriers, diluents or excipients.

The term “Lupus erythematosus” as used herein refers to is a category for a collection of diseases with similar underlying problems with immunity (autoimmune disease). Of these, systemic lupus erythematosus is the most common and serious form of lupus.

The term “lupus nephritis” as used herein refers to an inflammation of the kidney caused by systemic lupus erythematosus (SLE), a disease of the immune system. Apart from the kidneys, SLE can also damage the skin, joints, nervous system and virtually any organ or system in the body. Lupus nephritis, a typical autoimmune disorder with kidney involvement, is totally different from the so-called renal diseases.

In one example of the present invention, twelve-week-old New Zealand black/white (NZB/W) F1 lupus-prone mice were treated daily with EGCG by gavage until sacrificed at 34-weeks-old for clinical, pathological, and mechanistic evaluation. It was found that the administration (1) prevented proteinuria and renal function impairment and severe renal lesions, and (2) reduced renal oxidative stress, leukocyte infiltration, NF-κB activation, and NLRP3 mRNA/protein expression and serum levels of IL-1β and IL-18; (3) increased renal nuclear factor E2-related factor 2 (Nrf2) and glutathione peroxidase activity; (4) inhibited renal posttranslational processing of the precursors of caspase-1, IL-1β, and IL-18; and (5) enhanced regulatory T (Treg) cell activity. Our data clearly demonstrated that EGCG had prophylactic effects on lupus nephritis in these mice that were highly associated with its effects of enhancing the Nrf2 antioxidant signaling pathway, decreasing renal NLRP3 inflammasome activation, and increasing systemic Treg cell activity. Accordingly, the present invention provides a method for treating lupus nephritis, comprising administrating a subject in need thereof a therapeutically effective amount of showing an impaired oxidative status and increased levels of interleukin (IL)-1β and IL-18, which were closely linked to inflammation and correlated with disease activity. Although epigallocatechin-3-gallate (EGCG), the major bioactive polyphenol presents in green tea with antioxidant and free radical scavenging activities, has been reported to have anti-inflammatory effects by inhibiting nuclear factor-kappa B (NF-κB)-mediated inflammatory responses in vivo, its effectiveness for the treatment of lupus nephritis is still unknown.

In one particular example of the invention, EGCG has a preventive (prophylactic) effect in the development of lupus nephritis because EGCG was proved to have local effects in the kidney.

In other words, the present invention provides a new use of EGCG for manufacturing a medicament for preventing lupus nephritis development. In addition, the invention provides a pharmaceutical composition for treatment of lupus nephritis comprises a therapeutically effective amount of EGCG or a pharmaceutically acceptable salt or a physiologically functional derivative, together with one or more pharmaceutically acceptable carriers, diluents or excipients.

The term “pharmaceutically acceptable salt” as used herein refers to any non-toxic salt of the compound of the present invention. Typically, the pharmaceutically acceptable salts of the present invention may include acid addition salts. Representative salts include acetate, benzenesulfonate, benzoate, bicarbonate, bisulfate, bitartrate, borate, bromide, calcium edetate, camsylate, carbonate, chloride, clavulanate, citrate, hydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methyInitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. In one particular example of the invention, the pharmaceutically acceptable salt is hydrochloride.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation.

The term “therapeutically effective amount” as used herein refers to an amount of a drug or pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

For use in therapy, therapeutically effective amounts of ECGC, or a pharmaceutically acceptable salt or a physiological functional derivative thereof, may be formulated as a pharmaceutical composition for administration. Accordingly, the invention further provides a pharmaceutical composition comprising a therapeutically effective amount of the compound of Formula I and a pharmaceutically acceptable salt or a physiologically functional derivative thereof, and one or more pharmaceutically acceptable carriers, diluents, or excipients. The carrier(s), diluent(s) or excipient(s) must be acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation.

A therapeutically effective amount of ECGC will depend upon a number of factors. For example, the age and weight of the animal, the precise condition requiring treatment and its severity, the nature of the formulation, and the route of administration are all factors to be considered. For example, a therapeutically effective amount of a compound of Formula I for the treatment of humans suffering from type-2 diabetes, generally, should be in the range of 50 to 2000 mg per day, in one or several doses.

According to the present invention, the pharmaceutical composition may be adapted for administration by any appropriate route, including but not limited to oral, rectal, nasal, topical, vaginal, or parenteral route. In one particular example of the invention, the pharmaceutical composition is formulated for oral administration. Such formulations may be prepared by any method known in the art of pharmacy.

The present invention is further illustrated by the following examples, which are provided for the purpose of demonstration rather than limitation.

### Example

### Materials and Methods

Animal Model and Experimental Protocol

Female NZB/W F1 mice were purchased from the Animal Center of the National Taiwan University (Taipei, Taiwan, ROC) and, at 12 weeks of age, were divided into two groups, one of which was given normal saline and served as the disease control, while the other was given 120 mg/kg body weight of EGCG (Ryss Lab, Inc., CA, USA) (this dose is a slight modification of those used in previous in vivo studies) by gavage daily till sacrificed at 34-weeks-old. 8-Week-old NZB/W F1 female mice (prior to onset of autoantibody production) were used as normal controls. Urine samples were collected in metabolic cages every 2 weeks and urine protein levels were measured, and serum samples were used to measure serum levels of blood urea nitrogen (BUN) and creatinine (Cr) as described previously (Ka, S. M. et al. Decoy receptor 3 ameliorates an autoimmune crescentic glomerulonephritis model in mice. J Am Soc Nephrol 18:2473-2485; 2007). All animal experiments were performed upon the ethical approval of the Institutional Animal Care and Use Committee of the National Defense Medical Center, Taiwan and according to the ethical rules in the NIH Guide for the Care and Use of Laboratory Animals.

Pathologic Evaluation

Formalin-fixed and paraffin-embedded renal sections were prepared for pathologic evaluation.

For immune complex detection, frozen renal tissues were prepared and incubated with fluorescein isothiocyanate (FITC)-conjugated goat anti-mouse IgG antibodies (Cappel, N.C., USA). For immunohistochemistry (IHC), formalin-fixed and paraffin-embedded renal sections were prepared and incubated with primary antibodies against mouse CD3 (pan-T cell; Serotec, N.C., USA), F4/80 (monocyte/macrophage; Serotec), or NF-κB p65 (Cell Signaling Technology, MA, USA), then with biotinylated second antibodies (Dako, Glostrup, Denmark) and avidin-biotin-peroxidase complex (Dako). Scoring of staining was recorded.

Measurement of Serum Anti-DsDNA Antibodies

Serum levels of anti-dsDNA antibodies were measured using an anti-mouse dsDNA enzyme-linked immunosorbent assay (ELISA) kit (Alpha Diagnostic, TX, USA) according to the manufacturer's instructions. The absorbance at 450 nm was measured using an ELISA plate reader (Bio-Tek, VT, USA).

Measurement of Reactive Oxygen Species (ROS)

Kidney in situ superoxide anion production was determined by dihydroethidium (DHE) labeling. Fluorescent images were quantified by counting the percentage of positive nuclei in the total nuclei per kidney cross-section. Superoxide anion levels in serum and kidney tissues were measured as described previously and the results expressed as reactive luminescence units (RLU) per 15 min per milligram dry weight (i.e., RLU/15 min/mg dry weight).

Measurement of Cellular Glutathione Peroxidase (GPx) Activity in the Kidney

Renal cortex was lysed using RIPA lysis solution (Upstate Biotechnology, MA, USA). GPx activity in renal cortex lysates was measured using a commercial GPx assay kit (Cayman, Mich., USA) according to the manufacturer's instructions and enzyme activity expressed relative to the protein concentration in the lysate.

Western Blot Analysis

Cytoplasmic and nuclear proteins were extracted from renal cortex lysates using a Nuclear Extract Kit (Active Motif, Tokyo, Japan) according to the manufacturer's instructions and target proteins measured by Western blot analysis using rabbit antibodies against mouse Nrf2, p47^{phlox}, NLRP3, IL-18, caspase-1 (all from Santa Cruz Biotechnology, CA, USA), or IL-1β (US Biological, MA, USA). Antibodies to histone H3 (Cell Signaling) or β-actin (Santa Cruz) were used as internal controls for the nuclear and cytosolic target proteins, respectively.

Measurement of NLRP3 mRNA

Renal cortex RNA was extracted using TRIzol reagent (Invitrogen, CA, USA) according to the manufacturer's instructions. Real-time PCR was performed on an ABI Prism 7700 Sequence Detection System (Applied Biosystems, Foster City, Calif.) as described previously [42]. All of the probes and primers were Assays-on-Demand Gene expression products (Applied Biosystems). Amplifications were normalized to GAPDH using the 2^{-ΔCT} method.

Measurement of Serum IL-1β and IL-18

Serum levels of IL-1β and IL-18 were measured using commercial ELISA kits (BD Biosciences) according to the manufacturer's instructions.

Measurement of Renal NF-κB p65 Activation

Nuclear protein from renal cortex was extracted using a Nuclear Extract Kit (Active Motif) according to the manufacturer's instructions and nuclear NF-κB p65 activation quantified using an ELISA-based TransAM NF-κB kit (Active Motif, Tokyo, Japan) according to the manufacturer's protocol by reading the absorbance on an ELISA plate reader (Bio-Tek, VT, USA) at 450 nm with a reference wavelength of 655 nm.

Analysis of T/B Cell Activation

Mice splenocytes, isolated as described previously (Ka, S. M. et al. Decoy receptor 3 ameliorates an autoimmune crescentic glomerulonephritis model in mice. J Am Soc Nephrol 18:2473-2485; 2007), were stained for T or B cell activation with FITC-conjugated antibodies against mouse CD3 (17A2) or CD19 (1D3) and phycoerythrin (PE)-conjugated anti-mouse CD69 antibodies (H1.2F3) (all from BD Biosciences, CA, USA). Flow cytometric analysis was carried out using a FACSCalibur (BD Biosciences).

Treg Cell Functional Assay

The assay was performed using a published method (Nitcheu-Tefit, J. et al. Listeriolysin 0 expressed in a bacterial vaccine suppresses CD4+CD25high regulatory T cell function in vivo. J Immunol 179:1532-1541; 2007). Single cell suspensions were obtained from the spleens as described previously and CD4⁺ T cells negatively selected and fractionated into CD4⁺CD25⁻ and CD4⁺CD25⁺ subsets by magnetic Ab cell sorting (MACS, Bergisch Gladbach, Germany) using PE-labeled anti-CD25 mAb followed by anti-PE microbeads, according to the manufacturer's instructions. The purity of the cells was checked by FACS analysis and >90% of the CD4⁺ cells were shown to be either CD25⁻ or CD25⁺. The CD4⁺CD25⁺ T cells were used as Treg cells, while the CD4⁺CD25⁻ cells were added back to the original non-CD4 T cells and the mixture used as the responder T cells. For proliferation assays of the isolated components, 100×10³ responder T cells or Treg cells were cultured in RPMI 1640 medium containing 10% FCS (HyClone, IL, USA), 50 μM 2-ME (Sigma, Mo., USA), and 0.5 μg/ml of purified anti-CD3 mAb (BD Biosciences) in the presence of 200×10³ naive irradiated syngeneic splenocytes. In Treg cell functional assays, various numbers of Treg cells were added to 100×10³ responder cells, then the cells were cultured for 2 days with anti-CD3 mAb and splenocytes as above and proliferation measured by adding 1 μCi of ³H-methylthymidine (Amersham Pharmacia Biotech, NJ, USA) to each well for the last 18 h of culture, harvesting the cells and measuring thymidine incorporation using a TopCount (Packard, PerkinElmer, MA, USA) as described previously [42].

Data Analysis

The results are presented as the mean±SEM. Comparisons between two groups were performed using Student's t test. A p value of <0.05 was considered statistically significant.

Results

Clinical and Pathological Features

As shown in FIG. 1A, urine protein was measured every 2 weeks. Untreated disease control mice showed increased urine protein levels starting at week 28 and these continued to rise until the mice were sacrificed at week 34. These effects were markedly inhibited in the EGCG-treated mice, although they still showed mild proteinuria compared to normal controls. Likewise, a dramatic improvement in renal function was noted with EGCG treatment, as demonstrated by significantly lower serum levels of BUN (FIG. 1B) and Cr (FIG. 1C) in the EGCG-treated mice compared to the disease control mice at week 34.

When histopathological examination was performed on kidney sections (FIG. 1D), disease control mice showed diffuse glomerular proliferation, crescents, interstitial inflammation featuring mainly periglomerular mononuclear leukocyte infiltration, and interstitial fibrosis in combination with tubular atrophy with protein casts. These severe renal lesions were greatly reduced in EGCG-treated mice, although the mice still showed mild glomerular proliferation and mild interstitial inflammation.

Since autoantibody-induced immune complex deposition in the kidneys is considered to be the primary cause of lupus nephritis, we measured IgG deposits in the kidney and anti-dsDNA autoantibody levels in the serum. As shown in FIG. 1E, IgG immune complex deposition in the glomerulus was significantly higher in the disease control mice than in normal control mice, but there was no significant difference between the disease control and the EGCG-treated mice. In line with the IgG deposition results, the disease control and EGCG-treated mice had significantly higher serum anti-dsDNA antibody levels than normal control mice and again this effect was not inhibited by EGCG treatment (FIG. 1F).

ROS Production, GPx Activity, and Nrf2 Signaling Pathway

ROS production and Nrf2-mediated antioxidant activity were measured.

Reduction in ROS Levels in Serum and Urine

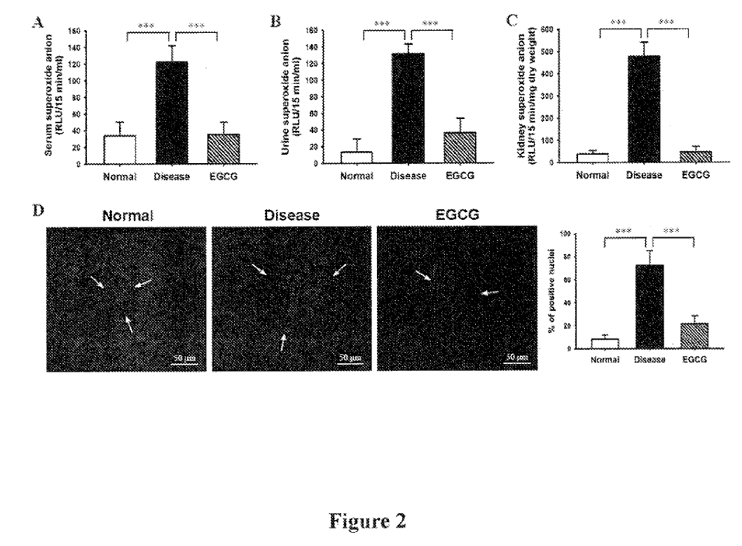

Disease control mice showed significantly increased superoxide anion levels in the serum (FIG. 2A) and urine (FIG. 2B) which were decreased by EGCG treatment to levels similar to those in normal control mice.

Inhibition of Renal ROS Production

Disease control mice had high levels of superoxide anion in the kidney and EGCG treatment inhibited this effect (FIG. 2C). To more specifically detect local ROS production in the kidney, in-situ ROS production in renal tissue was examined using the DHE assay. As shown in FIG. 2D, DHE fluorescence was low in the normal mouse kidney and significantly increased in disease control mice, showing increased in-situ ROS production compared to normal mice. In contrast, very low DHE fluorescence was observed in EGCG-treated mice.

Reduction of Renal Nad(P)H Oxidase Production and Activation of Renal Nrf2-Mediated Antioxidant Signaling Pathway

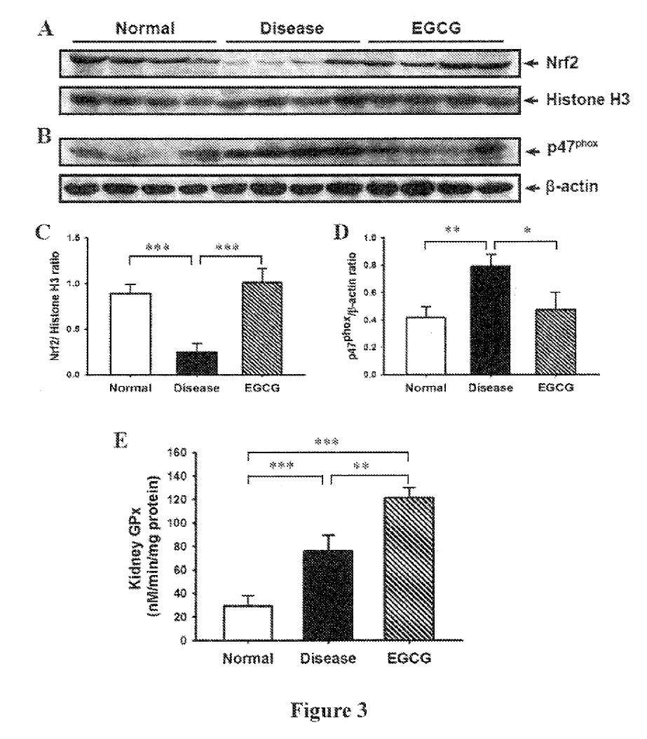

To determine whether EGCG administration affected antioxidative signaling, we measured Nrf2 translocation into nuclei (activation) and NAD(P)H oxidase subunit p47^{phox} levels and GPx activity in the kidney. As shown in FIGS. 3A and C, Nrf2 protein levels in nuclei were significantly lower in disease control mice than in normal controls, whereas EGCG-treated mice showed markedly increased Nrf2 translocation into the nucleus compared to disease controls, the level being similar to that in normal controls. In contrast, levels of NAD(P)H oxidase subunit p47^{phlox} were significantly increased in disease control mice and this effects was abolished by EGCG treatment (FIGS. 3B and D).

Furthermore, as shown in FIG. 3E, a marked increase in the activity of GPx, one of the phase II enzymes downstream of Nrf2, was seen in disease control mice, with an even larger increase in the EGCG-treated mice.

Renal Infiltration by T Cells and Monocytes/Macrophages

Infiltration of T cells and macrophages into the kidney plays an important role in rapidly progressive glomerular nephritis. Since EGCG has been shown to have antioxidant activities and to resolve inflammation in cisplatin-induced mice nephrotoxicity. Whether intra-renal infiltration of T cells was examined and monocytes/macrophages were found to be suppressed by EGCG administration. As demonstrated by IHC, compared to disease control mice, which showed marked infiltration of T cells (CD3⁺) (FIG. 4A) and monocytes/macrophages (F4/80⁺) (FIG. 4B) into the periglomerular region of the renal interstitium, EGCG-treated mice showed a similar pattern to normal controls.

Then, NF-κB activation in kidney tissues was measured. As demonstrated by IHC in FIG. 4C, NF-κB p65 activity was significantly increased in the kidney in disease control mice compared to normal controls, as shown by its nuclear location in the glomeruli and tubulointerstitium, and this effect was markedly inhibited in EGCG-treated mice. Consistent with the IHC results, ELISA results for renal tissue nuclear protein extracts showed that nuclear NF-κB p65 levels were significantly increased in disease control mice and that this effect was significantly inhibited by EGCG administration (FIG. 4D).

Serum Levels of IL-β and IL-18

Whether EGCG treatment affected serum levels of the proinflammatory cytokines IL-1β and IL-18 was examined by ELISA. As shown in FIGS. 5A and B, a marked increase in serum levels of these cytokines was seen in disease control mice compared to the low baseline levels observed in normal control mice, and levels in EGCG-treated mice were significantly lower than those in disease control mice.

NLRP3 Inflammasome Activation

NLRP3 mRNA and protein levels in kidney tissues was measured using real-time PCR and Western blotting, respectively. In disease control mice, levels of NLRP3 mRNA (FIG. 5C) and protein (FIGS. 5D and H) were significantly increased compared to normal controls and these effects were inhibited in EGCG-treated mice.

Whether inflammasome activation was further examined, and it was found to be suppressed by EGCG administration by measuring the posttranslational processing of procaspase-1 and the precursor forms of the proinflammatory cytokines IL-1β and IL-18. As demonstrated by Western blot analysis, increasing amounts of mature IL-1β (FIGS. 5E and I) and IL-18 (FIGS. 5F and J) were observed in lysates of disease control kidneys, but not EGCG-treated or normal control kidneys. Consistent with cytokine maturation, caspase-1 activation (FIGS. 5G and K) was also seen in disease control kidneys, as demonstrated by the appearance of the p20 subunit, and this effect was significantly inhibited by EGCG treatment.

Systemic Cellular Immunity and Treg Cell Activation

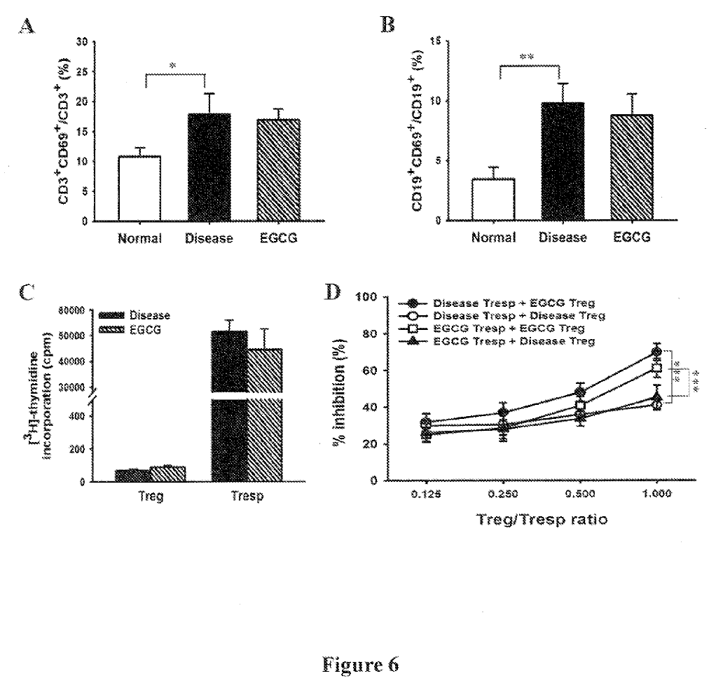

Whether EGCG treatment affected systemic immunity was examined by using splenocytes. As shown in FIG. 6A, the percentage of CD3⁺CD69⁺ cells (activated T cells) in CD3⁺ splenocytes was significantly increased in disease control mice compared to normal controls and this effect was not inhibited by EGCG. Similarly, the percentage of CD19⁺CD69⁺ cells (activated B cells) in CD19⁺ splenocytes was significantly increased in disease control mice and, although the percentage tended to decrease in the EGCG-treated mice compared to disease mice, there was no significant difference between these two groups (FIG. 6B).

Treg cells were proved to be protective against lupus nephritis by maintaining immune self-tolerance and EGCG has been shown to increase the number and promote the function of Treg cells in humans in vitro, the effect of EGCG on Treg cell function was investigated by performing titration studies in which different numbers of isolated CD4⁺CD25⁺ Treg cells were incubated with a fixed number of responder T cells (CD4⁺CD25⁻ T cells plus non-CD4 T cells) and stimulating the cultures with an anti-CD3 monoclonal antibody. As shown in FIG. 5C, when incubated alone, Treg cells from either disease control or EGCG-treated mice did not proliferate following anti-CD3 antibody stimulation, whereas responder T cells from either group of mice proliferated, but to an equal extent. As shown in FIG. 5D, the proliferation of responder T cells from disease control or EGCG-treated mice was reduced in a dose-dependent manner by addition of Treg cells from disease control mice and this inhibition was much greater using Treg cells from EGCG-treated mice, suggesting that mice treated with EGCG have increased Treg cell activity.

In conclusion, EGCG was proved to be protective in SLE and might be useful in therapy aimed at preventing the development of SLE, particularly lupus nephritis.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

We claim:

1. A method for regulating immune comprising administering a subject in need thereof a therapeutically effective amount of EGCG or a pharmaceutically acceptable salt or a physiologically functional derivative, together with one or more pharmaceutically acceptable carriers, diluents or excipients.

2. A method for preventing or treating lupus erythematosus comprising administering a subject in need thereof a therapeutically effective amount of EGCG or a pharmaceutically acceptable salt or a physiologically functional derivative, together with one or more pharmaceutically acceptable carriers, diluents or excipients.

3. A method of claim 2, wherein the lupus erythematosus is systemic lupus erythematosus (SLE).

4. A method of claim 2, wherein the lupus erythematosus is local lupus erythematosus.

5. A method of claim 4, wherein the local lupus erythematosus is lupus nephritis.

6. A method for preventing the development of lupus nephritis comprising administering a subject in need thereof a therapeutically effective amount of EGCG or a pharmaceutically acceptable salt or a physiologically functional derivative, together with one or more pharmaceutically acceptable carriers, diluents or excipients.
